(19) United States
(12) Patent Application Publication
(10) Pub. No.: US 2024/0327929 A1
(43) Pub. Date: Oct. 3, 2024

(54) DOSAGE REGIMEN FOR ADMINISTRATION OF BELZUTIFAN

(71) Applicants: Thomas J. BATEMAN, West Point, PA (US); Aparna CHHIBBER, Conshohocken, PA (US); Eunkyung KAUH, West Point, PA (US); Dhananjay Devidas MARATHE, Basking Ridge, NJ (US); Peter M. SHAW, West Point, PA (US); Rachel MARCEAU WEST, North Wales, PA (US); MERCK SHARP & DOHME LLC, RAHWAY, NJ (US)

(72) Inventors: Thomas J. BATEMAN, Doylestown, PA (US); Aparna CHHIBBER, Conshohocken, PA (US); Eunkyung KAUH, Philadelphia, PA (US); Dhananjay Devidas MARATHE, Basking Ridge, NJ (US); Peter M. SHAW, Yardley, PA (US); Rachel MARCEAU WEST, Holly Springs, NC (US)

(73) Assignee: MERCK SHARP & DOHME LLC, RAHWAY, NJ (US)

(21) Appl. No.: 18/681,539

(22) PCT Filed: Aug. 8, 2022
(86) PCT No.: PCT/US2022/039671

§ 371 Date: Feb. 6, 2024

Related U.S. Application Data
(60) Provisional application No. 63/232,415, filed on Aug. 12, 2021.

(51) Int. Cl. C12Q 1/6886; A61K 31/277; A61P 35/00; C12Q 1/6858

(52) U.S. Cl. CPC C12Q 1/6886; A61K 31/277; A61P 35/00; C12Q 1/6858; C12Q 2600/106

(57) ABSTRACT

The present disclosure provides a method of treating cancer or von Hippel-Lindau (VHL) disease with a safe and effective therapeutic dose of belzutifan in a patient in need thereof, comprising: (i) determining the belzutifan metabolic status (BMS) of the patient to determine whether the patient has a low metabolizer status, medium metabolizer status, or fast metabolizer status and (ii) (a) if the patient has the medium or fast metabolizer status, administering belzutifan to the patient at a standard therapeutic dose of 120 mg: or (ii) (b) if the patient has the low metabolizer status, administering belzutifan, to the patient at a therapeutic dose below that of the standard therapeutic dosc.

### FIELD OF THE INVENTION

The present disclosure relates to a dosage regimen suitable for administration of belzutifan which accounts for a patient's metabolic status. Such metabolic status may depend on, among other things, the patient's genotype for certain belzutifan metabolizing enzymes, the patient's bodyweight, and whether the patient is being administered a strong inhibitor of one of the metabolizing enzymes.

### BACKGROUND OF THE INVENTION

Intratumoral hypoxia is a driving force in cancer progression and is closely linked to poor patient prognosis and resistance to chemotherapy and radiation treatment. Hypoxia-Inducible Factors (HIF-1α and HIF-2α) are transcription factors that play central roles in the hypoxic response pathway. Under normoxic conditions, the tumor suppressor von Hippel-Lindau (VHL) protein binds to specific hydroxylated proline residues and recruits the E3 ubiquition-ligase complex that targets HIF-α proteins for proteasomal degradation. Under hypoxic conditions, HIF-α proteins accumulate and enter the nucleus to stimulate the expression of genes that regulate anaerobic metabolism, angiogenesis, cell proliferation, cell survival, extracellular matrix remodeling, pH homeostasis, amino acid and nucleotide metabolism, and genomic instability. VHL deficiency can also result in accumulated HIF expression under oxygenated conditions (pseudohypoxic conditions). Accordingly, directly targeting HIF-α proteins offers an exciting opportunity to attack tumors on multiple fronts (Keith, et al., Nature Rev. Cancer 12: 9-22, 2012).

Specifically, HIF-2α is a key oncogenic driver in clear cell renal cell carcinoma (ccRCC) (Kondo, K., et al., Cancer Cell, 1:237-246 (2002); Maranchie, J. et al, Cancer Cell, 1:247-255 (2002); Kondo, K., et al., PLOS Biol., 1:439-444 (2003)). In mouse ccRCC tumor models, knockdown of HIF-2α expression in pVHL (von Hippel-Lindau protein) defective cell lines blocked tumor growth comparable to reintroduction of p VHL. In addition, expression of a stabilized variant of HIF-2α was able to overcome the tumor suppressive role of p VHL.

Von Hippel-Lindau disease (VHL disease) is another disorder in which HIF-2α plays a significant role. VHL disease is an autosomal dominant syndrome that not only predisposes patients to kidney cancer (˜70% lifetime risk), but also to hemangioblastomas, pheochromocytoma and pancreatic neuroendocrine tumors. VHL disease results in tumors with constitutively active HIF-α proteins with the majority of these dependent on HIF-2 α activity (Maher, et al. Eur. J. Hum. Genet. 19: 617-623, 2011). HIF-2 α has been linked to cancers of the retina, adrenal gland and pancreas through both VHL disease and activating mutations.

3-[(1S,2S, 3R)-2,3-Difluoro-1-hydroxy-7-methylsulfonyl-indan-4-yl]oxy-5-fluoro-benzonitrile (hereinafter, belzutifan or MK-6482), a novel HIF-2α inhibitor with excellent in vitro potency, pharmacokinetic profile and in vivo efficacy in mouse models, has shown encouraging outcomes in patients with advanced renal cell carcinoma (Xu, Rui, et al., J. Med. Chem. 62:6876-6893 (2019).

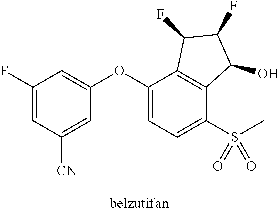 [SMILES: CS(=O)(=O)c1ccc(Oc2cc(F)cc(C#N)c2)c2c1[C@H](O)[C@H](F)[C@@H]2F]

In a recent report, belzutifan had a favorable safety profile and showed promising anti-tumor activity in heavily pre-treated patients with ccRCC. Choueiri, T. K. et al. i Nat Med 27, 802-805 (2021). In a dose-escalation cohort of the reported study, no dose-limiting toxicities occurred at doses up to 160 mg once daily, and the maximum tolerated dose was not reached. The recommended phase 2 dose was 120 mg once daily. The most common adverse events were anemia and hypoxia.

Belzutifan is generally well tolerated in human patients but different patients may metabolize the drug differently and thus, certain patients may need to be monitored for the side effects of anemia and hypoxia. These undesirable side effects may be ameliorated by dose titration regimens.

The effect of a given dose of belzutifan may be different in some patients than in others, for example, depending on how extensively the patient metabolizes belzutifan. It would therefore be desirable, in order to improve the risk benefit ratio for these patients, to be able to identify patients for whom these effects would be significantly different and adjust the treatment regimen accordingly.

### SUMMARY OF THE INVENTION

In one aspect, the present disclosure provides a method of treating cancer or von Hippel-Lindau (VHL) disease with a safe and effective therapeutic dose of belzutifan in a patient in need thereof, comprising:
(i) determining the belzutifan metabolic status (BMS) of the patient to determine whether the patient has a low metabolizer status, medium metabolizer status, or fast metabolizer status and
(ii) (a) if the patient has the medium or fast metabolizer status, administering belzutifan to the patient at a standard therapeutic dose of 120 mg; or
(ii) (b) if the patient has the low metabolizer status, administering belzutifan, to the patient at a therapeutic dose below that of the standard therapeutic dose.

### DETAILED DESCRIPTION OF THE INVENTION

### Definitions

Unless defined otherwise, all technical and scientific terms used herein have the same meaning as is commonly understood by one of skill in the art to which this invention belongs.

“Administering the therapeutic dose” of belzutifan means the dose is administered at the start of the treatment period following the determination of the patient's BMS, as defined below. The dose may be increased subsequently according to the judgment of the medical practitioner prescribing the belzutifan treatment.

“Amount”, “dose” or “dosage” of belzutifan as measured in milligrams refers to the milligrams of belzutifan (free form) present in a preparation, regardless of the form of the preparation.

“Allele” is a particular form of a gene or other genetic locus, distinguished from other forms by its particular nucleotide sequence,

“AUC” means the area under the concentration vs. time curve.

“Belzutifan metabolic status (BMS)” means the ability of a patient to metabolize belzutifan. The BMS can be determined based on a patient's UGTB17 and CYP2C19 phenotypes, body weight, whether the patient is being administered a strong UGTB17 inhibitor, a strong CYP2C19 inhibitor, and combinations of these properties or states.

“CYP2C19 poor metabolizer phenocopy” means a patient who is being administered a strong CYP2C19 inhibitor prior to being administered belzutifan. In some embodiments, the patient will under treatment of a therapeutic agent that is a strong CYP2C19inhibitor and will also be administered belzutifan. In some embodiments strong inhibitors of CYP2C19 are agents that increase the AUC of the sensitive index substrates of a metabolic pathway≥5 fold. Non-limiting examples of strong inhibitors of CYP2C19 include fluconazole, fluoxetine, fluvoxamine, and ticlopidine.

“Patient” means a human patient.

The term “patient in need thereof” as used herein refers to a patient diagnosed with or suspected of having von Hippel-Lindau disease or cancer as disclosed herein.

“Phenocopy” means an individual, e.g., a patient, showing features characteristic of a genotype other than its own, but produced environmentally rather than genetically. For instance, an individual that shows the features of a genotype of a poor metabolizer of a particular metabolic enzyme (but does not have the genotype of poor metabolizer for the enzyme), that results from administration of a strong inhibitor of the metabolic enzyme is a phenocopy.

“Treat” or “Treating” means to administer a therapeutic agent, such as a composition containing belzutifan, internally or externally to an individual in need of the therapeutic agent. Individuals in need of belzutifan include individuals who have been diagnosed as having, or at risk of developing, a condition or disorder susceptible to treatment with belzutifan. Typically, belzutifan is administered in a therapeutically effective amount, which means an amount effective to produce one or more beneficial results. The therapeutically effective amount of belzutifan may vary according to factors such as the disease state, age, and weight of the patient being treated, and the sensitivity of the patient, e.g., ability to respond, to the therapeutic agent. Whether a beneficial or clinical result has been achieved can be assessed by any clinical measurement typically used by physicians or other skilled healthcare providers to assess the presence, severity or progression status of the targeted disease, symptom or adverse effect. Typically, a therapeutically effective amount of an agent will result in an improvement in the relevant clinical measurement(s) over the baseline status, or over the expected status if not treated, of at least 5%, usually by at least 10%, more usually at least 20%, most usually at least 30%, preferably at least 40%, more preferably at least 50%, most preferably at least 60%, ideally at least 70%, more ideally at least 80%, and most ideally at least 90%.

“UGT2B17 poor metabolizer phenocopy” means a patient who is being administered a strong UGT2B 17 inhibitor prior to being administered belzutifan. In some embodiments, the patient will under treatment of a therapeutic agent that is a strong UGT2B17 inhibitor and will also be administered belzutifan. In some embodiments strong inhibitors of UGT2B17 are agents that increase the AUC of the sensitive index substrates of a metabolic pathway ≥ 5 fold.

### Specific Embodiments of the Present Disclosure

In one aspect, the present disclosure provides a method of treating cancer or von Hippel-Lindau (VHL) disease with a safe and effective therapeutic dose of belzutifan in a patient in need thereof, comprising:
(i) determining the belzutifan metabolic status (BMS) of the patient to determine whether the patient has a low metabolizer status, medium metabolizer status, or fast metabolizer status and
(ii) (a) if the patient has the medium or fast metabolizer status, administering belzutifan to the patient at a standard therapeutic dose of 120 mg; or
(ii) (b) if the patient has the low metabolizer status, administering belzutifan, to the patient at a therapeutic dose below that of the standard therapeutic dose.

In one embodiment of the method, the patient has a bodyweight of 45 kg or less. In such an embodiment, the present disclosure provides:
(a) the patient is determined to have the low metabolizer status when the patient has:
   (i) a UGT2B17 PM and CYP2C19 PM phenotype,
   (ii) a UGT2B17 PM and CYP2C19 intermediate metabolizer (IM) phenotype; and
(b) the patient is determined to have the medium metabolizer status when the patient has:
   (i) a UGT2B17 PM and CYP2C19 extensive metabolizer (EM) phenotype,
   (ii) a UGT2B17 PM and CYP2C19 rapid metabolizer (RM) phenotype,
   (iii) a UGT2B17 PM and CYP2C19 ultra-rapid metabolizer (UM) phenotype,
   (iv) a UGT2B17 IM and CYP2C19 PM phenotype,
   (v) a UGT2B17 IM and CYP2C19 IM phenotype,
   (vi) a UGT2B17 IM and CYP2C19 EM phenotype,
   (vii) a UGT2B17 IM and CYP2C19 RM phenotype,
   (viii) a UGT2B17 IM and CYP2C19 UM phenotype,
   (ix) a UGT2B17 EM and CYP2C19 PM phenotype,
   (x) a UGT2B17 EM and CYP2C19 IM phenotype,
   (xi) a UGT2B17 EM and CYP2C19 EM phenotype,
   (xii) a UGT2B17 EM and CYP2C19 RM phenotype; or
   (xiii) a UGT2B17 EM and CYP2C19 UM phenotype.

In embodiments of the method, wherein the patient has a bodyweight of 45 kg or less and only the phenotype of the UGT2B17 enzyme is determined, the present disclosure provides:
(a) the patient is determined to have the low metabolizer status when the patient has:
   a UGT2B17 poor metabolizer (PM) phenotype; and
(b) the patient is determined to have the medium metabolizer status when the patient has:
   (i) a UGT2B17 intermediate metabolizer (IM) phenotype, or
   (ii) a UGT2B17 extensive metabolizer (EM) phenotype.

In embodiments of the method, the patient has a bodyweight of 45 kg or less and only the phenotype of the CYP2C19 enzyme of the patient is determined, the present disclosure provides:
(a) the patient is determined to have the low metabolizer status when the patient has:
   a CYP2C19 poor metabolizer (PM) phenotype; and
(b) the patient is determined to have the medium metabolizer status when the patient has:
   (i) a CYP2C19 intermediate metabolizer (IM) phenotype, or
   (ii) a CYP2C19 extensive metabolizer (EM) phenotype.

In a specific embodiment of this method, the patient is from an east Asia state (e.g., Japan, China, Taiwan, Korea).

In another embodiment of the method, the patient has a bodyweight of 110 kg or more. In such an embodiment, the present disclosure provides:
(a) the patient is determined to have the low metabolizer status when the patient has
   a UGT2B17 poor metabolizer (PM) and CYP2C19 PM phenotype;
(b) the patient is determined to have the medium metabolizer status when the patient has:
   (i) a UGT2B17 PM and CYP2C19 intermediate metabolizer (IM) phenotype,
   (ii) a UGT2B17 PM and CYP2C19 intermediate metabolizer (IM) phenotype,
   (iii) a UGT2B17 PM and CYP2C19 extensive metabolizer (EM) phenotype,
   (iv) a UGT2B17 PM and CYP2C19 rapid metabolizer (RM) phenotype,
   (v) a UGT2B17 PM and CYP2C19 ultra-rapid metabolizer (UM) phenotype,
   (vi) a UGT2B17 IM and CYP2C19 PM phenotype,
   (vii) a UGT2B17 IM and CYP2C19 IM phenotype,
   (viii) a UGT2B17 IM and CYP2C19 EM phenotype,
   (ix) a UGT2B17 IM and CYP2C19 RM phenotype,
   (x) a UGT2B17 IM and CYP2C19 UM phenotype,
   (xi) a UGT2B17 EM and CYP2C19 PM phenotype,
   (xii) a UGT2B17 EM and CYP2C19 IM phenotype, or
   (xiii) a UGT2B17 EM and CYP2C19 EM phenotype, and
(b) the patient is determined to have the fast metabolizer status when the patient has a
   (i) a UGT2B17 EM and CYP2C19 RM phenotype, or
   (ii) a UGT2B17 EM and CYP2C19 UM phenotype.

In another embodiment of the method, the patient is being administered a strong UGT2B17 inhibitor (i.e., is a UGT2B17 poor metabolizer phenocopy) and has a bodyweight of greater than 45 kg. In such an embodiment, the present disclosure provides:
(a) the patient is determined to have the low metabolizer status when the patient has:
   a CYP2C19 poor metabolizer (PM) phenotype, and
(b) the patient is determined to have the medium metabolizer status when the patient has:
   (i) a CYP2C19 intermediate metabolizer (IM) phenotype,
   (ii) a CYP2C19 extensive metabolizer (EM) phenotype,
   (iii) a CYP2C19 rapid metabolizer (RM) phenotype, or
   (iv) a CYP2C19 ultra-rapid metabolizer (UM) phenotype.

In another embodiment of the method, the patient is being administered a strong inhibitor of CYP2C19 (i.e., is a CYP2C19 poor metabolizer phenocopy). The strong inhibitor of CYP2C19 can be, for example, selected from fluconazole, fluoxetine, fluvoxamine, or ticlopidine. In such an embodiment, the present disclosure provides:
(a) the patient is determined to have the low metabolizer status when the patient has:
   a UGT2B17 poor metabolizer (PM) phenotype; and
(b) the patient is determined to have the medium metabolizer status when the patient has:
   (i) a UGT2B17 intermediate metabolizer (IM) phenotype,
   (ii) a UGT2B17 extensive metabolizer (EM) phenotype.

In some embodiments of the method, the present disclosure provides that step (i), determining the BMS comprises:
(a) obtaining a biological sample (e.g., a blood sample) from the patient;
(b) detecting which copies of alleles of UGT2B17 and CYP2C19 are present in the biological sample; and
(c) determining the patient's UGT2B17 and CYP2C19 phenotypes from the detection of the UGT2B17 and CYP2C19 alleles.

In embodiments of the method wherein the phenotype of the UGT2B17 enzyme is determined, the present disclosure provides that step (i), determining the BMS comprises:
(a) obtaining a biological sample (e.g., a blood sample) from the patient;
(b) detecting which copies of alleles of UGT2B17 are present in the biological sample; and (c) determining the patient's UGT2B17 phenotype from the detection of the UGT2B17 alleles.

In embodiments of the method wherein the phenotype of the CYP2C19 enzyme is determined, the present disclosure provides that step (i), determining the BMS comprises:
(a) obtaining a biological sample (e.g., a blood sample) from the patient;
(b) detecting which copies of alleles of CYP2C19 are present in the biological sample; and
(c) determining the patient's CYP2C19 phenotype from the detection of the CYP2C19 alleles.

In certain embodiments of the invention, the patient having the UGT2B17PM tests positive for UGT2B17 *2/*2.

In some embodiments of the invention, the patient having the UGT2B17 IM tests positive for UGT2B17 *1/*2.

In certain embodiments of the invention, the patient having the UGT2B17 EM tests positive for UGT2B17 *1/*1.

In certain embodiments of the invention, the patient having the CYP2C19 PM tests positive for two CYP2C19 alleles selected from the group consisting of *2, *3, *4, *5, *6, *7, *8, *9 and *35.

In some embodiments of the invention, the patient having the CYP2C19 IM phenotype:
(a) tests positive for at least one CYP2C19 *1 allele and one of CYP2C19 alleles selected from the group consisting of *2, *3, *4, *5, *6, *7, *8, *9 and *35; or
(b) tests positive for at least one CYP2C19 *17 allele and one of CYP2C19 allele selected from the group consisting of *2, *3, *4, *5, *6, *7, *8, *9 and *35.

In certain embodiments of the invention, the patient having the CYP2C19 RM tests positive for CYP2C19 *1/*17.

In some embodiments of the invention, the patient having the CYP2C19 UM tests positive for CYP2C19 *17/*17.

In specific embodiments of the method, the present disclosure provides that the therapeutic dose below that of the standard therapeutic dose administered to the patient is 40 mg or 80 mg. In one embodiment the therapeutic dose below that of the standard therapeutic dose is 80 mg. In another embodiment the therapeutic dose below that of the standard therapeutic dose is 40 mg.

In some embodiments of the method, the patient is in need of treatment for cancer, for example, for the treatment of renal cell carcinoma (e.g., clear cell renal cell carcinoma).

In other embodiments of the method, the patient is in need of treatment of von Hippel-Lindau (VHL) disease. In specific embodiments, the patient is in need of treatment for VHL disease-associated renal cell carcinoma, central nervous system hemangioblastomas, or pancreatic neuroendocrine tumors, not requiring immediate surgery.

In a second aspect, the present disclosure provides a method of treating cancer or von Hippel-Lindau (VHL) disease with a safe and effective therapeutic dose of belzutifan in a patient in need thereof, comprising:
(i) determining the belzutifan metabolic status (BMS) of the patient to determine whether the patient has a low metabolizer status, medium metabolizer status, or fast metabolizer status and
(ii) (a) if the patient has the medium metabolizer status, administering belzutifan to the patient at a standard therapeutic dose of 120 mg;
(ii) (b) if the patient has the low metabolizer status, administering belzutifan, to the patient at a therapeutic dose below that of the standard therapeutic dose; or
(ii) (c) if the patient has the fast metabolizer status, administering belzutif an, to the patient at a therapeutic dose higher than the standard therapeutic dose (e.g., 160 mg).

### Belzutifan-Containing Pharmaceutical Compositions

Belzutifan may be administered any form, including oral solid and liquid dosage forms. Oral solid dosage forms are preferred dosage forms for administration in the methods of the invention. Preferred solid oral dosage forms include those disclosed in WO2020/092100, which can contain belzutifan in a solid dispersion and one or more pharmaceutically acceptable excipients, as a capsule or a tablet. The solid dispersion comprises a pharmaceutically acceptable polymer, which may be HPMCAS. Preferred dosage forms are tablets containing 40 mg of belzutifan.

Belzutifan can be prepared using processes disclosed in U.S. application Ser. No. 17/017,864, filed Sep. 11, 2020.

### Disorders for Treatment

The methods disclosed herein are useful for treating von Hippel-Lindau (VHL) disease or cancer.

In one embodiment, the present disclosure provides a method of treating VHL disease.

In a specific embodiment, the patient is in need of treatment for VHL disease-associated renal cell carcinoma, central nervous system hemangioblastomas, or pancreatic neuroendocrine tumors, not requiring immediate surgery.

In another embodiment, the present disclosure provides a method of treating cancer. In some embodiments, the cancer is selected from the group consisting of bladder cancer, breast cancer, non-small cell lung cancer (NSCLC), colorectal cancer (CRC), renal cell carcinoma (RCC), hepatocellular carcinoma (HCC), pancreatic cancer and melanoma.

In certain embodiments, the cancer is metastatic. In some embodiments, the cancer is relapsed. In other embodiments, the cancer is refractory. In yet other embodiments, the cancer is relapsed and refractory.

In one embodiment, the cancer is bladder cancer. In another embodiment, the cancer is breast cancer. In yet another embodiment, the cancer is NSCLC. In still another embodiment, the cancer is CRC. In one embodiment, the cancer is RCC. In another embodiment, the cancer is HCC. In yet another embodiment, the cancer is pancreatic cancer. In yet another embodiment, the cancer is melanoma.

In one embodiment, the cancer is advanced RCC. In another embodiment, the RCC is advanced RCC with clear cell component (ccRCC). In yet another embodiment, the cancer is metastatic RCC. In yet another embodiment, the cancer is relapsed RCC. In still another embodiment, the cancer is refractory RCC. In yet still another embodiment, the cancer is relapsed and refractory RCC.

In one embodiment, the human patient has not received prior systemic treatment for advanced disease. In a class of the embodiment, the human patient has not received prior systemic treatment for advanced RCC.

In one embodiment, the human patient has received prior systemic treatment for advanced disease.

### Patient Phenotype Determination

The genotype of the patient plays an important role in determining the observed phenotype, i.e., the observed capacity of the UGT2B17 and CYP2C19 enzymes to metabolize belzutifan. For the avoidance of doubt, in an aspect of the present disclosure, the patient phenotype is determined or inferred from the genotype.

The UGT2B17 phenotype may also be determined by administering a probe substrate of UGT2B17 and calculating a metabolic ratio (-plasma concentration of metabolite/parent compound). Similarly, the CYP2C19 phenotype may be determined by administering a probe substrate of CYP2C19 and calculating a second metabolic ratio (-plasma concentration of metabolite/parent compound).

Testing of the patient genotype for patients may be carried out by any standard testing method e.g., by a standard genotyping method, e.g., PCR assays, genomic arrays or, for example, sequencing DNA. The patient genotype may be determined by an in vitro test method e.g., a genotyping method. For example, in vitro testing may be carried out by taking biological sample, e.g., a body fluid (e.g., blood or saliva e.g., blood) or tissue sample from the patient and analyzing the sample by any standard testing method (e.g., PCR assays, genomic arrays or, for example, sequencing DNA) to determine the patient genotype. In an embodiment, the patient genotype is determined by analysis of a blood, saliva or tissue sample taken from the patient In a preferred embodiment, the patient genotype is determined by analysis of blood samples taken from the patient.

### EXAMPLE

The following example is provided to more clearly describe the present invention and should not be construed to limit the scope of the invention.

### Example 1

### Pharmacogenetic Analysis-Analysis of Phase 1 Studies to Evaluate the Effect of Selected Genetic Variants On Belzutifan Pharmacokinetics

Throughout the Example as well as the rest of the specification and claims, abbreviations and acronyms may be used with the following meanings unless otherwise indicated:

| Symbol | Definition |
|---|---|
| AUC | Area under the concentration versus time curve (h × ng/mL) |
| BID | Twice daily |
| BMI | Body mass index (kg/m²) |
| CI | Confidence interval |
| C_{MAX} | Peak plasma concentration (ng/mL) |
| C_{MIN} | Trough plasma concentration (ng/mL) |
| df | Degree of freedom |
| DNA | Deoxyribonucleic acid |
| EM | Extensive metabolizer, carrying no altered function alleles |
| | assayed |
| GMR | Geometric mean ratio |
| h | Hour |
| IM | Intermediate metabolizer, containing one loss of function or one |
| | or two reduced function alleles |
| kg | Kilogram |
| MAF | Minor allele frequency |
| MD | Multi-dose |
| mL | Milliliter |
| ng | Nanogram |
| NA | Not applicable |
| PCR | Polymerase chain reaction |
| PGx | Pharmacogenetic |
| PK | Pharmacokinetic |
| PK/PD | Pharmacokinetic/Pharmacodynamic |
| PM | Poor metabolizer, carrying two loss of function alleles |
| QD | Once daily |
| RCC | Renal cell carcinoma |
| RM | Rapid metabolizer, carrying one increased function allele and no |
| | reduced/loss of function alleles |
| SAP | Statistical analysis plan |
| SD | Single dose |
| SNP | Single nucleotide polymorphism |
| UM | Ultra-rapid metabolizer, carrying two increased function alleles |

### SUMMARY

Belzutifan is metabolized primarily by glucuronidation, catalyzed predominantly by UDP Glucuronosyltransferase Family 2 Member B17 (UGT2B17). It is additionally metabolized by oxidative metabolism, catalyzed by cytochrome P450 enzyme 2C19 (CYP2C19) and to a lesser extent CYP3 A4. A common deletion in UGT2B17 results in complete loss of UGT2B17 protein and corresponding loss of enzyme activity. (Xue, Y. et al. Adaptive evolution of UGT2B17 copy-number variation. Am J Hum Genet 2008, 83, 337-346). In addition, genetic variants in CYP2C19 are known to both decrease and increase activity of the enzyme. (Scott, S. A. et al. PharmGKB summary: very important pharmacogene information for cytochrome P450, family 2, subfamily C, polypeptide 19. Pharmacogenet Genom 2012, 22, 159-165). It is possible that the pharmacokinetics of belzutifan may be altered in subjects carrying genetic variants known to alter function in one or both enzymes.

The primary objective of this analysis was to evaluate the association between UGT2B17 and CYP2C19 phenotype (as defined by genotype) and inter-individual variability in exposure to MK-6482. Additional exploratory and sensitivity analyses explored the relationship between other covariates and enzyme phenotype and estimated average exposures in different patient populations. These analyses pooled data from four Phase I studies: MK-6482-001 (PT2977-101), MK-6482-002 (PT2977-103), MK-6482-006 (PT2977-104), and MK-6482-007, with a total sample size of 152 independent subjects with at least one PK measurement and genotype information for at least one of UGT2B17 and/or CYP2C19, with 188 AUC and Cₘₐₓ observations used to characterize belzutifan PK.

Linear mixed effect model analyses were performed on the natural log transformed PK parameters from the four pooled Phase 1 studies, separately for AUC and Cₘₐₓ. The model contained fixed effects of natural log transformed dose, drug formulation (old or new), enzyme phenotype determined from genotype, relevant additional covariates, and a random subject effect. UGT2B17 phenotype was considered as a categorical variable with dummy encoding for each phenotype (intermediate metabolizers (IM), poor metabolizers (PM)) that differed from the extensive metabolizer (EM) category). Similarly, CYP2C19 metabolizer status was considered as a categorical variable with dummy encoding for each phenotype (PM, IM, rapid metabolizers (RM) and ultra-rapid metabolizers (UM)) that differed from the EM category. Relevant covariates were selected under the null hypothesis from the following: disease (healthy vs patient), body weight (in kg), age, gender, and dose-by-body weight and dose-by-formulation interactions. Final models used to generate tables in this Example include log (dose), formulation (old/new), body weight (kg), and enzyme phenotype as variables. Note that this model assumes a linear change in exposure with change in dose and body weight, as well as equal phenotype effect by formulation, dose and body weight.

To understand the differences in exposure between different genetic phenotype categories, least square means were computed for each UGT2B17 phenotype, each CYP2C19 phenotype, and for each combination UGT2B17/CYP2C19 phenotype. Fold changes in expected exposure between each phenotype category and its corresponding reference category were computed as a geometric mean ratio, taking the exponentiated difference in log(PK) compared to the reference. Similar analyses were also conducted separately within each UGT2B17 phenotype group to quantify the difference in exposure between CYP2C19 phenotype groups separately for each UGT2B17 phenotype. Additional exploratory and sensitivity analyses were conducted based on these models. Full details of the analysis models are provided below.

### Introduction, Rationale and Summary of Conclusions

The pharmacokinetics of belzutifan (MK-6482) may be altered in subjects with increased or decreased activity in UGT2B17 and/or CYP2C19 driven by genetic variation in the genes encoding these enzymes. The goal of this analysis was to determine the extent to which such variation contributes to inter-individual variability in the pharmacokinetics of belzutifan, as well as to use models developed to provide estimates of exposure in particular patient populations.

The UDP Glucuronosyltransferase Family 2 Member B17 (UGT2B17) and cytochrome P450 enzyme 2C19 (CYP2C19) contribute to the metabolism of belzutifan. A common deletion in UGT2B17, the *2 allele, results in complete loss of UGT2B 17 protein and corresponding loss of enzyme activity. (Xue, Y. et al. Adaptive evolution of UGT2B17 copy-number variation. Am J Hum Genet 2008, 83, 337-346). Individuals carrying two copies of the deletion (*2/*2), UGT2B17 “poor metabolizers” (PMs), have no UGT2B17 activity. Individuals carrying one copy of the deletion (*1/*2), UGT2B17 “intermediate metabolizers” (IMs), have reduced enzyme activity as compared to individuals with two functional copies (*1/*1), extensive metabolizers (EMs). The frequency of the deletion varies widely across populations, resulting in substantial differences in UGT2B17 phenotype frequencies (8-1). The poor metabolizer phenotype occursin ˜15% of a European ancestry (white) population, and ˜70% of an East Asian population.

Genetic variants in CYP2C19 are known to both decrease and increase activity of the enzyme. (Scott, S. A. et al. PharmGKB summary: very important pharmacogene information for cytochrome P450, family 2, subfamily C, polypeptide 19. Pharmacogenet Genom 2012, 22, 159-165). The combination of altered function alleles in an individual determines the CYP2C19 metabolizer phenotype, and thus expected enzyme activity, in a given individual. Five phenotypes are commonly defined—“poor metabolizers” (PMs) carry two loss of function alleles, “intermediate metabolizers” (IMs) carry one loss of function allele or one or two reduced function alleles, “rapid metabolizers” (RMs) carry one increased function allele and no loss-of-function or reduced function alleles, “ultra-rapid metabolizers” (UMs) carry two increased function alleles, and “extensive metabolizers” (EMs) carry no altered function alleles. The frequency of CYP2C19 phenotypes also varies across populations (8-1). The poor metabolizer phenotype occurs in ˜2% of a European ancestry population, and ˜13% of an East Asian population.

The pharmacokinetics of belzutifan may be altered in subjects with increased or decreased activity in UGT2B17 and/or CYP2C19 driven by genetic variation in the genes encoding these enzymes. The goal of this analysis is to determine the extent to which such variation contributes to inter-individual variability in the PK of belzutifan, as well as to use models developed to provide estimates of exposure in particular patient populations.

### Objectives

The primary objectives of this pharmacogenetics analysis were:
To investigate the differences in exposure to belzutifan between UGT2B17 PMs and IMs compared to EMs, as well as exposure differences between PMs compared to all other subjects (e.g., pooled IMs and EMs), controlling for CYP2C19 phenotype.
To investigate the difference in exposure to belzutifan between CYP2C19 PMs, IMs, RMs, and UMs compared to EMs, both overall, adjusting for UGT2B17 phenotype, and separately within each UGT2B17 phenotype.
To investigate the difference in exposure to belzutifan between subjects who carry different combinations of UGT2B17 and CYP2C19 phenotype, compared to double EMs (CYP2C19 and UGT2B17 EMs).
   The exploratory objectives of this PGx analysis were:
To evaluate any potential dependence between body weight and UGT2B17 activity.
To estimate the mean belzutifan exposure in each UGT2B17 and CYP2C19 phenotype category for subjects with body weights of 60 kg and 80 kg given 120 and 80 mg of the new formulation.
To estimate a population-level difference in average exposure of belzutifan between East Asian, Japanese, South Asian, and African ancestry subjects as compared to European ancestry subjects based on expected frequencies of UGT2B17 and CYP2C19 phenotypes in each population.
   The main conclusions of this analysis are as follows:
Exposure to MK-6482, as measured by area under the concentration vs time curve (AUC), is higher in individuals with no or reduced UGT2B17 activity. The GMR (95% CI) of AUC was 2.40 (2.03, 2.84) for poor metabolizers of UGT2B17 relative to extensive metabolizers, 1.55 (1.37, 1.75) for intermediate metabolizers relative to extensive metabolizers (Table 4-4), and 1.93 (1.43, 2.61) for poor metabolizers relative to the average of intermediate and extensive metabolizers (Table 4-5).
Exposure to MK-6482, as measured by AUC, is higher in individuals with reduced CYP2C19 activity, particularly among those individuals with no UGT2B17 activity (poor metabolizers). Among UGT2B17 poor metabolizers, the GMR (95% CI) of AUC for CYP2C19 poor metabolizers relative to CYP2C19 extensive metabolizers was 2.42 (1.96, 3.00) and 1.39 (1.13, 1.70) for CYP2C19 intermediate metabolizers relative to CYP2C19 extensive metabolizers (Table 4-7).
In subjects with reduced activity of both enzymes (double poor metabolizers), the GMR (95% CI) of AUC is 4.33 (3.32, 5.66) relative to extensive metabolizers for both enzymes when allowing for a differential CYP2C19 effect within different levels of UGT2B17 (i.e., including UGT2B17-by-CYP2C19 interaction effect) (Table 4-9).
In addition to enzyme phenotype, body weight was independently associated with MK-6482 AUC in this dataset, with lower exposure in heavier individuals. For every 10 kg increase in body weight a 9.2% decrease in AUC is expected on average. There is no evidence that the association between exposure and body weight is dependent on UGT2B17 activity in this dataset.
Assuming the same body weight in each population, the GMR for AUC (95% CI) is 1.82 (1.63, 2.04) for Japanese ancestry subjects, 1.66 (1.51, 1.83) for East Asian ancestry subjects, 1.29 (1.23, 1.35) for South Asian ancestry subjects, and 0.91 (0.89, 0.94) for African ancestry subjects as compared to European ancestry subjects (Table 4-11). Accounting for differences in body weight (i.e., assuming 60 kg average body weight for East Asians and Japanese subjects and 80 kg for Europeans), the GMR for AUC (95% CI) is 2.17 (1.95, 2.43) for Japanese subjects as compared to European ancestry subjects and 1.98 (1.80, 2.18) for East Asian subjects overall as compared to European ancestry subjects (Table 4-12).
Similar trends were observed for Cₘₐₓ as outlined for AUC, although with smaller magnitude of effect in all analyses. No evidence of association between CYP2C19 and Cₘₐₓ was observed, though trends indicate there may be a minor effect within UGT2B17 poor metabolizers.

### METHODS

### Subjects and Methods

### Clinical Data

Four phase I studies were included in this PGx analysis:
PT2977-101/MK-6482-001 is a dose escalation trial in subjects with renal cell carcinoma or advanced solid tumors. The trial was conducted in several parts: Part 1A was the dose escalation stage designed to identify the maximum tolerated dose. Part 1B and Part 2 were expansion cohorts designed to assess safety, PK, and preliminary efficacy at the selected dose (120 mg) from Part 1A.
PT2977-103/MK-6482-002 is a single dose (120 mg) food effect study conducted in healthy volunteers.
PT2977-104/MK-6482-006 is a three-way crossover study designed to assess bioavailability, safety, and pharmacokinetics (PK) of two formulations (120 mg old formulation, 120 mg new formulation, and 200 mg new formulation) of PT2977 in healthy volunteers.
MK-6482-007 is a single dose (40 mg) study to assess the pharmacokinetics of MK-6482 in Caucasian and Japanese healthy female volunteers with specified CYP2C19 phenotypes.

There are many important differences in patient composition between the trials. To note, MK-6482-001 was a first-in-human dose finding trial studying patients with advanced solid tumors or renal cell carcinoma (RCC), while MK-6482-002,-006, and-007 were PK trials conducted with (predominantly female) healthy volunteers. In addition, study-007 enrolled Japanese subjects based on specific CYP2C19 phenotypes; all other studies enrolled subjects without genotype-based selection criteria. Finally, the protocols differed in terms of belzutifan formulation given to trial subjects: subjects in protocols 001 and 002 were given one formulation of belzutifan, study 006 compared that formulation and a new formulations, enrolling all subjects to receive both the formulations, and study 007 patients only received the new formulation of MK-6482.

### Genetic Data

DNA from 174 appropriately consented subjects was extracted from peripheral blood samples and using the Affymetrix PharmacoscanTM array (studies-001,-002, and-006), using PCR-based assays (study 007). DNA was not available for 3 subjects in the PK dataset. Four samples failed quality control metrics used to assess sample quality (three from study-002 and one from study-001). Two subjects in study-002 were found to be genetically identical to two subjects in study-006, indicating that the same subjects enrolled in both studies (which was permitted by the Sponsor) or that these subjects were identical twins. For the purpose of this analysis we assumed the same subject enrolled in both studies. For two additional subjects, genotype for one or both enzymes could not be accurately determined using the data generated.

### PK Endpoints

The Phase 1 PK endpoints analyzed were AUCo->following single dose (SD) administration and steady state AUCo-, following multiple dose (MD) administration pooled, as well as Cmax following SD administration and steady state Cmax following MD administration pooled. Only PK parameter values following the administration of belzutifan at fasting state administered once daily (QD) were included in the analyses; all fed subjects from protocol-002 and all subjects receiving the 120 mg BID dose from protocol-001 were removed prior to analysis.

### Statistical Analysis

The analysis was performed according to the PGx statistical analysis plan.

### Primary Objectives

### Statistical Model for Primary Objectives

The following linear mixed effects model was fit to evaluate the relationship between UGT2B17 and CYP2C19 metabolizer phenotype and MK-6482 exposure:

$${Y}_{ij} = {β}_{0} + {β}_{1} log ( {dose}_{ij} ) + {β}_{2} {form}_{ij} + {β}_{3} {I}_{UGTPM} + {β}_{4} {I}_{UGTIM} + {β}_{5} {I}_{CYPPM} + {β}_{6} {I}_{CYPIM} + {β}_{7} {I}_{CYPRM} + {β}_{8} {I}_{CYPUM} + {\sum }_{ℓ = 1}^{p} {γ}_{ℓ} {X}_{u} + {S}_{i} + {ϵ}_{ij} \qquad ( 1 )$$

where Yᵢⱼ is the log-transformed exposure endpoint of interest (e.g., area under the plasma concentration time curve at steady state or maximum concentration) for measurement j of subject i, dose is the dosage of study drug received (in mg), log transformed to match evidence of dose proportionality across the studies at typical doses examined, form is the drug formulation (old vs new), and Xᵢₗ are additional covariates selected from candidate covariates: age, gender, disease status (healthy volunteer vs patient), weight, and interaction terms log(dose)-by-formulation and log(dose)-by-weight under the null model including no effect of UGT2B17 or CYP2C19. Between- and within-subject random effects were modeled via terms Sᵢ and ϵᵢⱼ, respectively, and assumed to be normally distributed.

UGT2B17 phenotype was coded categorically, allowing a non-linear relationship between poor metabolizers (PM), intermediate metabolizers (IM), and extensive metabolizers (EM). Similarly, CYP2C19 phenotype was coded categorically with 5 categories summarizing metabolizer status (PM, IM, EM, rapid metabolizer (RM), and ultra-rapid metabolizer (UM). For each phenotype group, dummy variables were used to measure change in exposure from extensive metabolizer category. Indicator variables Igeneg in Model (1) are equal to one for all subjects with gene (i.e., UGT2B17 or CYP2C19) metabolizer status g (e.g., PM, IM, RM, or UM) and zero for all other subjects.

### Covariate Selection for Primary Objectives

In model (1), additional covariates were selected from a candidate list including age, gender, disease status (healthy volunteer vs patient), weight, and interaction terms log(dose)-by-formulation and log(dose)-by-weight under the null model including no effect of UGT2B17 or CYP2C19. Lasso variable selection was performed using the glmmlasso R package, performing variable tuning using an AIC criterion. For AUC, weight was selected to be included in the model; for Cₘₐₓ, no additional covariates were selected, but weight was included in the model for consistency as discussed in the SAP. Stepwise selection was also considered as a sensitivity analysis. Using this approach, weight, age, and disease status were selected for AUC, and weight, disease status, and log(dose)-by-formulation interaction were selected for Cₘₐₓ. Final results were very similar between the two models, so we focus on the lasso-based results (e.g., including weight in the final models) in results below.

### Testing and Estimated Fold Changes

Global tests of association were performed between both UGT2B17 and CYP2C19 phenotypes and MK exposure using F-tests of the null hypotheses H_{0,UGT}:β₃=β₄=0 and H_{0,CYP}:β₅=β₆=β₇=δ₈=0, respectively using Kenward-Roger denominator degrees of freedom for the fixed effects.

To understand differences in exposure between different genetic phenotype categories, least square means were computed for each UGT2B17 phenotype, each CYP2C19 phenotype, and for each combination UGT2B17/CYP2C19 phenotype category. Fold changes in expected exposure were computed between each phenotype category and its corresponding reference category as a geometric mean ratio, calculated as the exponentiated difference in log(PK) compared to the reference. For UGT2B17, an additional contrast comparing poor metabolizers to the average of intermediate and extensive metabolizer exposure was also computed. For joint phenotype comparisons, fold changes were computed with reference to the double extensive metabolizer (EM/EM) subjects. For each contrast, 95% confidence intervals were computed referencing the t-distribution and t-tests were performed to get an estimate of significance. P-values were adjusted for multiple testing using a Bonferroni adjustment, accounting for the number of tests performed within each testing group (e.g., 3 UGT2B17 tests, 4 CYP2C19 tests, and 14 joint UGT2B17/CYP2C19 tests).

### Exploring Differential CYP2C19 Effect Within UGT2B17

In model (1) we did not consider any interactive effects between CYP2C19 and UGT2B17 phenotypes. However, the fraction eliminated by CYP2C19 is expected to be different for subjects with differing UGT2B17 phenotype. To explore this, we considered estimating fold change for each CYP2C19 metabolizer status compared to the extensive metabolizers separately within UGT2B17 poor, intermediate, and extensive metabolizers. Within each UGT2B17 phenotype, we fit a model of the form:

$${Y}_{ij} = {β}_{0}^{*} + {β}_{1}^{*} log ( {dose}_{ij} ) + {β}_{2}^{*} {form}_{ij} + {β}_{3}^{*} {I}_{CYPPM} + {β}_{4}^{*} {I}_{CYPIM} + {β}_{5}^{*} {I}_{CYPRM} + {β}_{6}^{*} {I}_{CYPUM} + {\sum }_{ℓ = 1}^{p} {γ}_{ℓ}^{*} {X}_{u} + {S}_{i} + {ϵ}_{ij} \qquad ( 2 )$$

where covariates were chosen to match those selected for model (1) (i.e., Xᵢ₁ is body weight in kg). Within each group, fold change, calculated via exponentiation of least square mean differences, and corresponding 95% confidence intervals referencing the t-distribution were calculated for each CYP2C19 metabolizer status non-wild-type category (e.g., poor metabolizers, intermediate metabolizers, rapid metabolizers, and ultra-rapid metabolizers) vs the extensive metabolizers. As there was some variability in these, we also considered a fully interactive model including all pairwise UGT2B17-by-CYP2C19 first order interaction terms. For this model,

CYP2C19 phenotype categories RM and UM were pooled as only 3 subjects were UMs and seemed to have consistent effects with RMs from the available trial data. Joint least square means and corresponding fold changes were recomputed using this interactive model to explore the sensitivity to the non-interactive assumption.

### Exploratory Objectives

### Exploring Effect of Body Weight and UGT2B17 Phenotype

In order to understand if the effect of UGT2B17 on MK-6482 exposure differs with weight, an extension of model (1) was fit including a body weight-by-UGT2B17 phenotype interaction term. Presence of an interactive effect was tested using an F-test with Kenward-Roger denominator degrees of freedom for the fixed effects. As a descriptive measure, the effect of body weight on exposure was also calculated separately within each UGT2B17 group using a mixed effects model accounting for log(dose) and formulation only. Estimated percent change in exposure per additional 10 kg body weight, along with corresponding 95% confidence intervals, was computed within each UGT2B17 category and overall. For the overall estimate, model (1) was used.

### Estimating Population Differences in Exposures

Estimated mean exposures were calculated for each of the following key genetic race groups of interest: European, East Asian, South Asian, African, and European ancestry subjects as the weighted mean of least square means for each joint UGT2B17 and CYP2C19 metabolizer phenotype category with weights corresponding to the population frequencies as given in 8-1. Estimated exposures were based on model (1) extended to include all first order UGT2B17-by-CYP2C19 interaction effects. Estimated fold change in MK-6482 exposure between each ancestry group and European subjects were computed as the geometric mean ratio, exponentiating the difference in least square means for East Asian/Japanese subjects and

European subjects assuming a fixed weight across subjects, as well as computed using reference weights of 60 kg for East Asian/Japanese subjects and 80 kg for European subjects.

### Software

RStudio running R version 3.6.0 x86_64 was used to complete this analysis.

### Results

### Analysis Population

The analysis population was composed of subjects pooled from the four Phase 1 studies that satisfied consent requirements and had both PK and genetic data available for analysis. 170 subjects across all studies were genotyped; 2 pairs of subjects were determined to be genetically identical and were treated as the same individual for the purpose of analysis. 6 subjects treated with MK-6482 twice daily (BID) were excluded from analyses. 10 subjects were excluded from analyses due to missing genetic or PK data. Model fits were conducted based on 188 observations in 152 subjects for both parameters (after accounting for the two duplicate subjects).

### Demographic and Phenotype Summary

Table 4-1 and 4-2 summarize the CYP2C19 and UGT2B17 phenotype information for all Phase 1 subjects analyzed (subjects with both PK and genetic data) by study (4-1) and across all subjects (4-2). Note that two subjects in study 002 were genetically identical to two subjects in 006 and were treated as the same individual in these analyses. Details regarding alleles used to determine phenotypes and their frequencies in the analysis dataset are included in 8-1.

TABLE 4-1
UGT2B17 and CYP2C19 phenotype for subjects
included in PGx analyses, by study.

| | Study | | | | |
|---|---|---|---|---|---|
| | 001 | 002 | 006 | 007 | Total |
| UGT2B17 Phenotype | | | | | |
| Extensive | 39 | 5 | 6 | 4 | 54 |
| Intermediate | 35 | 5 | 10 | 12 | 62 |
| Poor | 2 | 1 | 2 | 33 | 38 |
| CYP2C19 Phenotype | | | | | |
| Ultra-Rapid | 2 | 1 | 0 | 0 | 3 |
| Rapid | 16 | 3 | 2 | 3 | 24 |
| Extensive | 33 | 3 | 10 | 19 | 65 |
| Intermediate | 22 | 3 | 5 | 14 | 44 |
| Poor | 3 | 1 | 1 | 13 | 18 |
| Total | 76 | 11 | 18 | 49 | 154 |

TABLE 4-2
UGT2B17 and CYP2C19 phenotype for subjects included
in PGx analyses, across all subjects.

| CYP2C19 | UGT2B17 Phenotype | | | |
|---|---|---|---|---|
| Phenotype | Poor | Intermediate | Extensive | Total |
| Poor | 10 | 5 | 3 | 18 |
| Intermediate | 11 | 16 | 17 | 44 |
| Extensive | 15 | 27 | 22 | 64 |
| Rapid | 2 | 13 | 8 | 23 |
| Ultra-Rapid | 0 | 1 | 2 | 3 |
| Total | 38 | 62 | 52 | 152 |

### Demographic Summary

Table 4-3 summarizes relevant demographic information for subjects included in PGx analyses.

TABLE 4-3
Demographic summary by study for PGx analysis population.

| | Study | | | | |
|---|---|---|---|---|---|
| | 001 | 002 | 006 | 007 | Total |
| Disease | | | | | |
| Tumor/RCC | 76 | 0 | 0 | 0 | 76 |
| Healthy | 0 | 11 | 18 | 49 | 78 |
| Gender | | | | | |
| Male | 55 | 0 | 3 | 0 | 58 |
| Female | 21 | 11 | 15 | 49 | 96 |
| Race | | | | | |
| Asian | 0 | 0 | 1 | 36 | 37 |
| Black or African | 6 | 4 | 8 | 0 | 18 |
| American | | | | | |
| White | 70 | 7 | 9 | 13 | 99 |
| Total | 76 | 11 | 18 | 49 | 154 |

105 subjects across protocols 001, 002, and 006 received one MK-6482 formulation while 67 received a newer formulation, including 18 subjects from 006 who were also given the older formulation and those subjects enrolled in study 007.

### Phase 1 Analysis Results

### UGT2B17

There is significant evidence of association between UGT2B17 phenotype and exposure for both endpoints of interest (AUC F-test p=1.09× 10⁻¹⁸; Cₘₐₓ, F-test p=1.52×10⁻³). Tables 4-4 and 4-5 display the effect of UGT2B17 phenotypes on MK-6482 PK parameters. The GMR (95% CI) of AUC was 2.40 (2.03, 2.84) for PMs relative to EMs and 1.55 (1.37, 1.75) for IMs relative to EMs, and 1.93 (1.43, 2.61) for PMs relative to IMs+EMs. These results indicate an increase in AUC associated with reduced UGT2B17 activity. Similar conclusions can be drawn from the results for Cₘₐₓ.

TABLE 4-4
Association between UGT2B17 phenotype (PM vs
EM and IM vs EM) and MK-6482 PK parameters

| | GMR Estimate (95% CI) | |
|---|---|---|
| UGT2B17 Phenotype | AUC (h*ng/mL) | Cmax (ng/mL) |
| Poor (PM) | 2.40 (2.03, 2.84) *** | 1.30 (1.12, 1.52) ** |
| Intermediate (IM) | 1.55 (1.37, 1.75) *** | 1.16 (1.04, 1.29) * |
| Extensive (EM) | 1.00 | 1.00 |

GMR: Geometric mean ratio, representing fold change in geometric mean PK for each metabolizer status (referencing EM)
* Bonferroni adjusted p-value < 0.05; adjusted for 3 contrasts
** Bonferroni adjusted p-value < 0.01; adjusted for 3 contrasts
*** Bonferroni adjusted p-value < 0.001; adjusted for 3 contrasts

TABLE 4-5
Association between UGT2B17 phenotype (PM
vs IM + EM) and MK-6482 PK parameters

| | GMR Estimate (95% CI) | |
|---|---|---|
| UGT2B17 Phenotype | AUC (h*ng/mL) | Cmax (ng/mL) |
| Poor (PM) | 1.93 (1.43, 2.61) *** | 1.21 (0.92, 1.59) * |
| Intermediate (IM), | 1.00 | 1.00 |
| Extensive (EM) | | |

GMR: Geometric mean ratio, representing fold change in geometric mean PK for each metabolizer status (referencing subjects with at least one copy of UGT2B17, average of EM and IM)
* Bonferroni adjusted p-value < 0.05; adjusted for 3 contrasts
** Bonferroni adjusted p-value < 0.01; adjusted for 3 contrasts
*** Bonferroni adjusted p-value < 0.001; adjusted for 3 contrasts

### CYP2C19

There is significant evidence of association between CYP2C19 phenotype and MK-6482 AUC (F-test p=1.06 × 10-7). There is not sufficient evidence of an association between CYP2C19 phenotype and MK-6482 Cₘₐₓ (F-testp=0.355). phenotypes on MK-6482 PK parameters. Across all subjects, the GMR (95% CI) of AUC was 1.71 (1.43, 2.04) for PMs relative to EMs. A trend to higher exposure in IMs and lower exposure in RMs and UMs was observed, but these comparisons were not statistically significant. A test of UGT2B17-by-CYP2C19 interaction effect on MK-6482 exposure was not significant for either endpoint (AUC F-test p=0.193; Cₘₐₓ F-test p=0.748). However, when evaluated separately within each UGT2B17 phenotype group, a larger difference between CYP2C19 PMs and EMs (2.42 (1.96, 3.00)) and CYP2C19 IMs and EMs (1.52 (1.11, 2.09)) was observed among UGT2B17 PMs for AUC. In fact, a global test of association between CYP2C19 and AUC was significant only within UGT2B17 phenotypes (p=1.23 × 10⁻⁸, 0.069, and 0.077 within PMs, IMs, and EMs, respectively). Interaction was incorporated in future prediction models due to this observed trend and was kept for Cₘₐₓ for consistency despite no evidence of CYP2C19 effect within any UGT2B17 phenotype group (p=0.082, 0.548, and 0.755 within PMs, IMs, and EMs, respectively).

Tables 4-6 and 4-7 display the genetic effects of CYP2C19 phenotypes on MK-6482 PK parameters. Across all subjects, the GMR (95% CI) of AUC was 1.71 (1.43, 2.04) for PMs relative to EMs. A trend to higher exposure in IMs and lower exposure in RMs and UMs was observed, but these comparisons were not statistically significant. A test of UGT2B17-by-CYP2C19 interaction effect on MK-6482 exposure was not significant for either endpoint (AUC F-test p=0.193; Cₘₐₓ F-testp=0.748). However, when evaluated separately within each UGT2B17 phenotype group, a larger difference between CYP2C19 PMs and EMs (2.42 (1.96, 3.00)) and CYP2C19 IMs and EMs (1.52 (1.11, 2.09)) was observed among UGT2B17 PMs for AUC. In fact, a global test of association between CYP2C19 and AUC was significant only within UGT2B17 phenotypes(p=1.23×10^{-8, 0.069}, and 0.077 within PMs, IMs, and EMs, respectively). Interaction was incorporated in future prediction models due to this observed trend and was kept for Cₘₐₓ for consistency despite no evidence of CYP2C19 effect within any UGT2B17 phenotype group (p=0.082, 0.548, and 0.755 within PMs, IMs, and EMs, respectively).

TABLE 4-6
Association between CYP2C19 phenotype and
MK-6482 PK parameters across all subjects

| | GMR Estimate (95% CI) | |
|---|---|---|
| CYP2C19 Phenotype | AUC (h*ng/mL) | Cmax (ng/mL) |
| Poor (PM) | 1.71 (1.43, 2.04) *** | 1.14 (0.98, 1.34) |
| Intermediate (IM) | 1.09 (0.96, 1.23) | 1.00 (0.89, 1.11) |
| Extensive (EM) | 1.00 | 1.00 |
| Rapid (RM) | 0.89 (0.76, 1.05) | 1.09 (0.95, 1.25) |
| Ultra-rapid (UM) | 0.85 (0.58, 1.25) | 1.05 (0.75, 1.48) |

GMR: Geometric mean ratio, representing fold change in geometric mean PK for each metabolizer status (referencing EM)
* Bonferroni adjusted p-value < 0.05; adjusted for 4 contrasts
** Bonferroni adjusted p-value < 0.01; adjusted for 4 contrasts
*** Bonferroni adjusted p-value < 0.001; adjusted for 4 contrasts

TABLE 4-7
Association between CYP2C19 phenotype and MK-6482
PK parameters within each UGT2B17 phenotype group

| CYP2C19 Phenotype | UGT2B17 PM | UGT2B17 IM | UGT2B17 EM |
|---|---|---|---|
| | AUC GMR Estimate (95% CI) | | |
| Poor (PM) | 2.42 (1.96, 3.00) *** | 1.52 (1.11, 2.09) * | 1.38 (0.87, 2.21) |
| Intermediate (IM) | 1.39 (1.13, 1.70) ** | 1.08 (0.89, 1.33) | 1.01 (0.79, 1.28) |
| Extensive (EM) | 1.00 | 1.00 | 1.00 |
| Rapid (RM) | 0.78 (0.53, 1.16) | 0.96 (0.77, 1.19) | 0.77 (0.57, 1.04) |
| Ultra-rapid (UM) | N/A | 1.37 (0.71, 2.63) | 0.62 (0.36, 1.07) |
| | Cmax GMR Estimate (95% CI) | | |
| Poor (PM) | 1.25 (1.01, 1.54) | 1.10 (0.80, 1.51) | 1.05 (0.74, 1.48) |
| Intermediate (IM) | 1.14 (0.93, 1.39) | 0.99 (0.81, 1.20) | 0.93 (0.77, 1.12) |
| Extensive (EM) | 1.00 | 1.00 | 1.00 |
| Rapid (RM) | 1.4 (0.94, 2.07) | 1.1 (0.89, 1.36) | 1.01 (0.8, 1.27) |
| Ultra-rapid (UM) | N/A | 1.59 (0.82, 3.08) | 0.8 (0.52, 1.23) |

GMR: Geometric mean ratio, representing fold change in geometric mean PK for each metabolizer status (referencing EM)
* Bonferroni adjusted p-value < 0.05; adjusted for 4 contrasts
** Bonferroni adjusted p-value < 0.01; adjusted for 4 contrasts
*** Bonferroni adjusted p-value < 0.001; adjusted for 4 contrasts

### UGT2B17 and CYP2C19

Table 4-8 displays the genetic effects of the combination of UGT2B17 and CYP2C19 phenotypes as compared to subjects who do not carry altered function alleles of either enzyme (UGT2B17 EM+CYP2C19 EM). The GMR (95% CI) of AUC for subjects who are PMs for both enzymes is 4.09 (3.25, 5.15) relative to EMs for both enzymes. This value is similar when allowing an interactive UGT2B17-by-CYP2C19 effect on AUC: 4.33 (3.32, 5.66) (see Table 4-9). The GMR (95% CI) of AUC for UGT2B17 PM+CYP2C19 PM subjects relative to those who are both UGT2B17 (IM+EM) and CYP2C19 (IM+EM+RM+UM) is 3.81 (3.00, 4.83).

TABLE 4-8
Association between combined UGT2B17 and CYP2C19 phenotype
across all phenotype combinations and MK-6482 exposure, assuming
no UGT2B17-by-CYP2C19 interactive effect on exposure

| | UGT2B17 Phenotype | | |
|---|---|---|---|
| CYP2C19 Phenotype | Poor (PM) | Intermediate (IM) | Extensive (EM) |
| | AUC GMR Estimate (95% CI) | | |
| Poor (PM) | 4.09 (3.25, 5.15) *** | 2.65 (2.13, 3.29) *** | 1.71 (1.43, 2.04) *** |
| Intermediate (IM) | 2.61 (2.11, 3.22) *** | 1.69 (1.41, 2.02) *** | 1.09 (0.96, 1.23) |
| Extensive (EM) | 2.40 (2.03, 2.84) *** | 1.55 (1.37, 1.75) *** | 1.00 |
| Rapid (RM) | 2.14 (1.69, 2.72) *** | 1.39 (1.14, 1.69) * | 0.89 (0.76, 1.05) |
| Ultra-rapid (UM) | 2.05 (1.33, 3.15) * | 1.32 (0.87, 2.00) | 0.85 (0.58, 1.25) |
| | Cmax GMR Estimate (95% CI) | | |
| Poor (PM) | 1.49 (1.21, 1.84) ** | 1.33 (1.10, 1.60) | 1.14 (0.98, 1.34) |
| Intermediate (IM) | 1.30 (1.08, 1.57) | 1.16 (0.99, 1.35) | 1.00 (0.89, 1.11) |
| Extensive (EM) | 1.30 (1.12, 1.52) ** | 1.16 (1.04, 1.29) | 1.00 |
| Rapid (RM) | 1.42 (1.15, 1.75) * | 1.26 (1.06, 1.50) | 1.09 (0.95, 1.25) |
| Ultra-rapid (UM) | 1.37 (0.94, 2.01) | 1.22 (0.85, 1.76) | 1.05 (0.75, 1.48) |

GMR: Geometric mean ratio, representing fold change in geometric mean PK for each metabolizer status (referencing the double EM), assuming no UGT2B17-by-CYP2C19 interaction
* Bonferroni adjusted p-value < 0.05; adjusted for 14 contrasts
** Bonferroni adjusted p-value < 0.01; adjusted for 14 contrasts
*** Bonferroni adjusted p-value < 0.001; adjusted for 14 contrasts

TABLE 4-9
Association between combined UGT2B17 and CYP2C19 phenotype
across all phenotype combinations and MK-6482 exposure, assuming
a UGT2B17-by-CYP2C19 interactive effect on exposure

| | UGT2B17 Phenotype | | |
|---|---|---|---|
| CYP2C19 Phenotype | Poor (PM) | Intermediate (IM) | Extensive (EM) |
| | AUC GMR Estimate (95% CI) | | |
| Poor (PM) | 4.33 (3.32, 5.66) *** | 2.16 (1.56, 2.98) *** | 1.28 (0.86, 1.90) |
| Intermediate (IM) | 2.52 (1.95, 3.27) *** | 1.53 (1.24, 1.89) ** | 0.99 (0.81, 1.22) |
| Extensive (EM) | 2.02 (1.61, 2.53) *** | 1.41 (1.18, 1.70) ** | 1.00 |
| Rapid/Ultra-rapid | 1.87 (1.16, 3.00) | 1.39 (1.11, 1.73) * | 0.73 (0.57, 0.93) |
| (RM/UM) | | | |
| | Cmax GMR Estimate (95% CI) | | |
| Poor (PM) | 1.43 (1.12, 1.83) | 1.27 (0.94, 1.70) | 1.10 (0.79, 1.54) |
| Intermediate (IM) | 1.31 (1.03, 1.66) | 1.08 (0.9, 1.30) | 0.95 (0.79, 1.14) |
| Extensive (EM) | 1.18 (0.97, 1.45) | 1.11 (0.94, 1.30) | 1.00 |
| Rapid/Ultra-rapid | 1.66 (1.08, 2.55) | 1.24 (1.02, 1.51) | 0.95 (0.77, 1.19) |
| (RM/UM) | | | |

GMR: Geometric mean ratio, representing fold change in geometric mean PK for each metabolizer status (referencing the double EM), using joint model with all first order UGT2B17-by-CYP2C19 interactions
* Bonferroni adjusted p-value < 0.05; adjusted for 14 contrasts
** Bonferroni adjusted p-value < 0.01; adjusted for 14 contrasts
*** Bonferroni adjusted p-value < 0.001; adjusted for 14 contrast

### Impact of Non-Genetic Factors on Exposure

Our model suggests a nearly proportional effect of dose on exposure (AUC) with \hat{β}_{logdose}=0.95. The new formulation is expected to have about exp(\hat{β}_{form})=0.92 of the exposure of the old formulation. The estimated effects for Cₘₐₓ are similar with \hat{β}_{log(dose)}=0.90 and expected Cₘₐₓ of new formulation ˜0.79 that of the old formulation.

Due to the high level of confoundedness between covariates in the dataset and the strong correlation between enzyme phenotype and race, race was not tested as an independent covariate during variable selection and was not included in the primary analysis models. As an exploratory analysis, the residual effect of race on exposure after adjusting for key covariates of weight and joint phenotype was evaluated in the final model. A test of association of race and exposure, after accounting for weight and joint phenotype, was significant for this data set (AUC p=0.025; Cₘₐₓ p=0.003). However, race was confounded with body weight, disease status, and age in this analysis, all of which showed some indication of association with AUC during the stepwise variable selection process. We note that, after adjusting for the covariates of disease status and age as selected by the stepwise variable selection model, no further significant reduction in residual variability was observed with the addition of a race covariate.

Body weight was associated with exposure (AUC). For every 10 kg increase in body weight an 9.2 (6.5, 12.0) % decrease in AUC and a 7.0 (4.5, 9.5) % decrease in Cₘₐₓ is expected. Table 4-10 displays the association between body weight (kg) MK-6482 exposure by UGT2B17 phenotype group after accounting for dose and formulation. There was no notable difference in the association between body weight and exposure by UGT2B17 phenotype group. A test of the interaction between UGT2B17 phenotype and body weight was similarly not significant for either endpoint (AUC p=0.637; Cₘₐₓ p=0.713).

TABLE 4-10
Estimated percent change in AUC (h*ng/mL)
and Cmax (ng/mL) per 10 kg increase in body
weight (kg) by UGT2B17 phenotype group.

| | UGT2B17 PM | UGT2B17 IM | UGT2B17 EM |
|---|---|---|---|
| % Change in AUC | −15.5 | −8.3 | −10.9 |
| (h*ng/mL) | (−29.5, −1.5) | (−11.9, −4.6) | (−16.5, −5.4) |
| % Change in Cmax | −4.5 | −7.8 | −6.4 |
| (ng/mL) | (−13.7, 4.7) | (-11.4, −4.1) | (−10.4, −2.3) |

Percent change calculated from model accounting for dose, formulation

### Differences in Exposure Between Populations

Tables 4-11 and 4-12 display the difference in exposure between Japanese, East Asian, South Asian, and African ancestry subjects as compared to European ancestry subjects at a fixed body weight and for Japanese and East Asian ancestry subjects as compared to European ancestry subjects assuming a body weight of 80 kg for Europeans and 60 kg for East Asians and Japanese subjects, respectively. The average body weight for East Asian and Japanese populations overall (˜60 kg) was selected based on reported average body weights in the China Health and Nutrition Survey 2006-2011 (Yuan, S. et al. The association of fruit and vegetable consumption with changes in weight and body mass index in Chinese adults: a cohort study. Public Health 2018, 157, 121-126). (average for males 64.8 kg, females 56.9 kg ages 18-65) and the Japanese National Health and Nutrition Survey 2017 tables (average for males 59.0-69.7 kg, females 48.7-55.0 kg>20 years of age). (Ikeda, N., Takimoto, H., Imai, S., Miyachi, M. & Nishi, N. Data Resource Profile: The Japan National Health and Nutrition Survey (NHNS). Int J Epidemiol 2015, 44, 1842-1849). For reference, the average body weight for East Asian ancestry subjects in this dataset was ˜56 kg; note that all East Asian ancestry subjects were female. The average body weight for a European ancestry population (˜80 kg) was selected based on the average body weight in for all white subjects in the analysis dataset (˜83 kg). For reference, the average body weight for non-Hispanic white males in the United States from 2015-2016 was 91.7 kg and 77.5 kg for non-Hispanic white females in the United States for the same time period. (Fryar CD, K .-M. D., Gu Q, Ogden CL. Mean Body Weight, Height, Waist Circumference, and Body Mass Index Among Adults: United States, 1999-2000 Through 2015-2016. Natl Health Stat Report. 2018, 122, 1-16). Population exposure estimates are derived from the expected exposures of each enzyme phenotype weighted by their expected frequencies in each population (Appendix Tables 8-4 to 8-6). At a fixed body weight, the GMR for AUC (95% CI) is 1.82 (1.63, 2.04) for Japanese ancestry subjects, 1.66 (1.51, 1.83) for East Asian ancestry subjects, 1.29 (1.23, 1.35) for South Asian ancestry subjects, and 0.91 (0.89, 0.94) for African ancestry subjects as compared to European ancestry subjects. Allowing for differences in body weight, the GMR for AUC (95% CI) is 2.17 (1.95, 2.43) for Japanese ancestry subjects as compared to European ancestry subjects and 1.98 (1.80, 2.18) for East Asian ancestry subjects overall as compared to European ancestry subjects.

TABLE 4-11
Estimated population level fold change in MK-6482 exposure,
assuming the same body weight in each population.

| Population | AUC GMR Estimate (95% CI) | Cmax GMR Estimate (95% CI) |
|---|---|---|
| Japanese | 1.82 (1.63, 2.04) *** | 1.13 (1.02, 1.26) |
| East Asian | 1.66 (1.51, 1.83) *** | 1.11 (1.02, 1.22) |
| South Asian | 1.29 (1.23, 1.35) *** | 1.07 (1.03, 1.12) ** |
| African | 0.91 (0.89, 0.94) *** | 0.96 (0.94, 0.98) ** |
| European | 1.00 | 1.00 |

GMR: Geometric mean ratio, representing fold change in geometric mean PK for each population, referencing European subjects, assuming fixed body weight
* Bonferroni adjusted p-value < 0.05; adjusted for 4 contrasts
** Bonferroni adjusted p-value < 0.01; adjusted for 4 contrasts
*** Bonferroni adjusted p-value < 0.001; adjusted for 4 contrasts

TABLE 4-12
Estimated population level fold change in MK-6482 exposure, assuming different
body weights for East Asian/Japanese and European ancestry subjects

| Population | AUC GMR Estimate (95% CI) | Cmax GMR Estimate (95% CI) |
|---|---|---|
| Japanese | 2.17 (1.95, 2.43) *** | 1.30 (1.17, 1.45) *** |
| East Asian | 1.98 (1.8, 2.18) *** | 1.28 (1.17, 1.41) *** |
| European | 1.00 | 1.00 |

GMR: Geometric mean ratio, representing fold change in geometric mean PK for each population, referencing European subjects, assuming 60 kg body weight for East Asians, Japanese and 80 kg for Europeans
* Bonferroni adjusted p-value < 0.05; adjusted for 2 contrasts
** Bonferroni adjusted p-value < 0.01; adjusted for 2 contrasts
*** Bonferroni adjusted p-value < 0.001; adjusted for 2 contrasts

### Genotype Deviations

Based on additional genetic analyses conducted after the primary genetic analysis dataset was generated, two subjects potentially had incorrect phenotype assignment. Subject 114 in study MK-6482-001 carries one copy of the UGT2B17*2 allele (and thus one copy of UGT2B17) and is classified as a UGT2B17 IM in the primary analysis dataset. After sequencing this subject, we determined that they carry one copy of the rare rs7548683 15 variant; this variant is predicted to alter splicing resulting in a non-functional UGT2B17 protein, suggesting that this subject may be better classified as a PM. The variant is very rare, occurring in approximately 1 in 20,000 European ancestry individuals. Similarly, Japanese ancestry subject 2 in study MK-6482-007 is classified as a CYP2C19 EM (*1/*1) in the primary analysis dataset; the increased function * 17 allele was not genotyped as part of the initial genotyping panel for Japanese ancestry subjects in this study. Subsequent genotyping of MK-6482 subjects on the PharmacoscanTM genotyping array used for other trials and a targeted PCR based assay revealed that this subject is a CYP2C19 RM (*1/* 17). Sensitivity analyses were conducted for the effect estimates for UGT2B17 and CYP2C19 phenotype using the updated phenotype definitions for these two subjects. These results were very similar to the primary analysis results, suggesting that mis-classification of these subjects did not meaningfully impact the results of the primary analyses.

### Discussion

Exposure to MK-6482 is significantly higher in individuals with reduced UGT2B17 activity, with PMs of the enzyme having over two-fold (2.40 (95% CI: 2.03, 2.84)) higher exposure than EMs after accounting for differences in body weight and CYP2C19 phenotype. Exposure to MK-6482 also appears to be somewhat higher in subjects with reduced CYP2C19 activity, with PMs of the enzyme having 1.71-fold (95% CI: 1.43, 2.04) higher exposure than EMs after accounting for differences in body weight and UGT2B17 phenotype. While a test of UGT2B17-by-CYP2C19 phenotype interactions was not statistically significant, an analysis of CYP2C19 phenotype effect separately within each UGT2B17 phenotype group showed a trend to increasing impact of reduced activity of CYP2C19 among subjects with reduced UGT2B17 activity, consistent with increasing relative contribution of CYP2C19 to the metabolism of MK-6482 in subjects with reduced UGT2B17 activity. A trend to decreased AUC among rapid and ultra-rapid metabolizers of CYP2C19 was observed, although this effect was not statistically significant overall or in any UGT2B17 phenotype group. Only 3 ultra-rapid metabolizers of CYP2C19, in whom the largest increase in CYP2C19 activity is expected, were enrolled in this study, and as such this dataset was not well powered to evaluate the impact of ultra-rapid metabolizers on exposure. That said, we expect the lowest exposures to occur in subjects who are ultra-rapid metabolizers of CYP2C19 and extensive metabolizers of UGT2B17; since the contribution of CYP2C19 to MK-6482 metabolism is expected to be the smallest in UGT2B17 extensive metabolizers, we do not expect genotype-driven exposures to be substantially lower than currently estimated.

In individuals who are PMs of both enzymes, a further increase in exposure compared to PMs of only one enzyme was observed in this study; model-based estimates suggest that PMs of both enzymes would have over four-fold higher exposure than EMs of both enzymes. PMs of both enzymes represent up to 15% (Table 8-6) of a Japanese ancestry population and 9% of an East Asian ancestry population (Table 8-5). While PMs of both enzymes occur relatively infrequently in a European ancestry population, such individuals will be present in the population, and notably one European ancestry poor metabolizer of both enzymes was observed in this dataset. The impact of enzyme phenotype on Cₘₐₓ showed similar trends to those observed for AUC, although with smaller effect sizes. We note that this analysis only considered variants in both enzymes with known impact on activity; it is possible there are additional variants associated with enzyme activity and exposure. In particular, while the impact of variants in CYP2C19 have been studied in depth in the literature, research on variants in UGT2B17 other than the *2 allele (large deletion) is more limited, and it is likely there is additional genetic variation in UGT2B17 that is associated with activity.

In addition to enzyme phenotypes, body weight was also found to contribute to variability in AUC, with a 9.2% decrease in AUC expected for every 10 kg increase in body weight, assuming a linear relationship between body weight and exposure. There was some indication that age and disease status may also be associated with AUC and disease status with Cₘₐₓ, however these variables were not selected by the lasso regularization approach used for variable selection for the final models. Gender was not independently associated with AUC or Cmax in this dataset. Because of the design of trials included in this analysis, a number of clinical and demographic factors were strongly correlated with each other, and as such it may not be possible to accurately identify independent effects of each of these non-genetic factors with the data available. For example, gender was correlated with both formulation and dose, and thus it is possible there is an impact of gender on exposure that we were not able to capture in this analysis.

Several assumptions were made about the relationship between dose and exposure in these analyses; in particular we assumed no interaction between enzyme phenotype and dose and between formulation and dose and assumed a linear relationship between dose and exposure. There was some indication that there may be an interaction between formulation and dose for Cₘₐₓ, with lower Cₘₐₓ with the new formulation at higher doses of MK-6482, however this interaction term was not selected by the lasso variable selection approach and was not included in the final models.

Results from the final models including only weight, log(dose), formulation, CYP2C19 phenotype and UGT2B17 phenotype as covariates were very similar to more complex models including additional terms (age and disease statusfor AUC and disease status and log(dose)*formulation for Cₘₐₓ). The fixed effects from our final predictive model collectively explained about 73% of the variability in the data in log(AUC), and about 76% of the variability in log(Cₘₐₓ).

The range of body weights observed in this dataset was 41 kg to 164 kg. A ˜2.9-fold difference in exposure between individuals at these extremes of body weight is expected, independent of any difference in exposure driven by enzyme phenotype.

Because the frequency of both UGT2B17 and CYP2C19 phenotypes varies between populations, particularly between East Asian populations and other groups, average exposure to MK-6482 is expected to vary between populations. Average AUCs in each population were calculated based on the least-square mean estimates for each pairwise phenotype group, then combined based on the frequency of each phenotype group in the population. The average AUC of MK-6482 in a Japanese ancestry population is estimated to be approximately double the exposure in a European ancestry population, with slightly larger differences if allowing for expected differences in body weight between populations. Note that population phenotype frequencies are estimates based on available datasets but do vary from study to study; this uncertainty in the phenotype frequencies is not incorporated into exposure estimates. As such, any estimates made based on population phenotype frequencies should be treated as approximate values.

### Overall Conclusions

The main conclusions of this analysis are as follows:
Exposure to MK-6482, as measured by area under the concentration vs time curve (AUC), is higher in individuals with no or reduced UGT2B17 activity. The GMR (95% CI) of AUC was 2.40 (2.03, 2.84) for poor metabolizers of UGT2B17 relative to extensive metabolizers, 1.55 (1.37, 1.75) for intermediate metabolizers relative to extensive metabolizers (Table 4-4), and 1.93 (1.43, 2.61) for poor metabolizers relative to the average of intermediate and extensive metabolizers (Table 4-5).
Exposure to MK-6482, as measured by AUC, is higher in individuals with reduced CYP2C19 activity, particularly among those individuals with no UGT2B17 activity (poor metabolizers). Among UGT2B17 poor metabolizers, the GMR (95% CI) of AUC for CYP2C19 poor metabolizers relative to CYP2C19 extensive metabolizers was 2.42 (1.96, 3.00) and 1.39 (1.13, 1.70) for CYP2C19 intermediate metabolizers relative to CYP2C19 extensive metabolizers (Table 4-7).
In subjects with reduced activity of both enzymes (double poor metabolizers), the GMR (95% CI) of AUC is 4.33 (3.32, 5.66) relative to extensive metabolizers for both enzymes when allowing for a differential CYP2C19 effect within different levels of UGT2B17 (i.e., including UGT2B17-by-CYP2C19 interaction effect) (Table 4-9).
In addition to enzyme phenotype, body weight was independently associated with MK-6482 AUC in this dataset, with lower exposure in heavier individuals. For every 10 kg increase in body weight a 9.2% decrease in AUC is expected on average. There is no evidence that the association between exposure and body weight is dependent on UGT2B17 activity in this dataset).
Assuming the same body weight in each population, the GMR for AUC (95% CI) is 1.82 (1.63, 2.04) for Japanese ancestry subjects, 1.66 (1.51, 1.83) for East Asian ancestry subjects, 1.29 (1.23, 1.35) for South Asian ancestry subjects, and 0.91 (0.89, 0.94) for African ancestry subjects as compared to European ancestry subjects (Table 4-11). Accounting for differences in body weight (i.e., assuming 60 kg average body weight for East Asians and Japanese subjects and 80 kg for Europeans), the GMR for AUC (95%

CI) is 2.17 (1.95, 2.43) for Japanese subjects as compared to European ancestry subjects and 1.98 (1.80, 2.18) for East Asian subjects overall as compared to European ancestry subjects (Table 4-12).
Similar trends were observed for Cₘₐₓ as outlined for AUC, although with smaller magnitude of effect in all analyses. No evidence of association between CYP2C19 and Cₘₐₓ was observed, though trends indicate there may be a minor effect within UGT2B17 poor metabolizers.

### APPENDICES

### Definition and Population Frequencies of UGT2B17 and CYP2C19 phenotypes

Sources for frequencies cited below include: the 1000 Genomes Project (1000 Genomes Project. A global reference for human genetic variation. Nature 2015, 526, 68-74), PharmGKB (Whirl-Carrillo, M. et al. Pharmacogenomics knowledge for personalized medicine. Clin Pharmacol Ther 2012 92, 414-417), and J Clin Pharmacol. 2010 August; 50(8):929-40.

TABLE 8-1
CYP2C19 and UGT2B17 allele definition for alleles assayed in this analysis

| | | | | Expected Allele Frequency (%) * | | | | |
|---|---|---|---|---|---|---|---|---|
| | | | Functional | | | African/ | | |
| | | | Impact on | | East | African | South | Admixed |
| Gene | Allele | Definition | activity | European | Asian | American | Asian | American |
| CYP2C19 | *1 | Absence of all | “Normal” | 62.73 | 60.66 | 59.00 | 47.46 | 69.48 |
| | | alleles below | Function | | | | | |
| | *2 | 19154G > A (Splice | No | 14.60 | 29.00 | 18.30 | 34.30 | 12.50 |
| | | Defect, P227P) | Function | | | | | |
| | *3 | 17948G > A(W212X) | No | 0.62 | 8.32 | 0.26 | 0.94 | 0.28 |
| | | | Function | | | | | |
| | *17 [symbol] | −806C > T | Increased | 21.30 | 1.31 | 20.00 | 17.30 | 15.30 |
| | | | Function | | | | | |
| UGT2B17 | *2 | — | “Normal” | 63.22 | 17.36 | 75.57 | 39.87 | 65.56 |
| | | | Function | | | | | |
| | | Deletion | No | 36.78 | 82.64 | 24.43 | 60.13 | 34.44 |
| | | | Function | | | | | |

* CYP2C19 frequencies and phenotype definitions from PharmGKB (https://www.pharmgkb.org/page/cyp2c19RefMaterials); UGT2B17*2 frequencies from 1000 Genomes Phase 3v5 [symbol]
The CYP2C19*17 allele was not assayed in Japanese subjects in study MK6482-007

TABLE 8-2
CYP2C19 phenotype definition and expected frequency

| | | Expected Phenotype Frequency (%) * | | | | | |
|---|---|---|---|---|---|---|---|
| | | | East | | African | Central/South | |
| Phenotype | Definition | European | Asian | Japanese | American | Asian | Latino |
| Ultra-rapid | Individuals | 4.68 | 0.04 | NA | 4.29 | 2.92 | 2.88 |
| metabolizer: | carrying two | | | | | | |
| UM | increased | | | | | | |
| | function | | | | | | |
| | alleles of | | | | | | |
| | CYP2C19 | | | | | | |
| Rapid | Individuals | 27.22 | 2.53 | NA | 23.74 | 18.57 | 24.53 |
| Metabolizer: | carrying one | | | | | | |
| RM | increased | | | | | | |
| | function | | | | | | |
| | allele of | | | | | | |
| | CYP2C19 | | | | | | |
| | and no | | | | | | |
| | reduced or | | | | | | |
| | loss of | | | | | | |
| | function | | | | | | |
| | alleles. | | | | | | |
| Extensive | Individuals | 39.56 | 38.40 | 32.4 | 33.03 | 29.55 | 52.24 |
| Metabolizer: | carrying no | | | | | | |
| EM | altered | | | | | | |
| | function | | | | | | |
| | alleles of | | | | | | |
| | CYP2C19. | | | | | | |
| Intermediate | Individuals | 26.15 | 46.01 | 48.4 | 34.18 | 40.81 | 19.19 |
| Metabolizer: | carrying one | | | | | | |
| IM | or two | | | | | | |
| | reduced or | | | | | | |
| | one loss-of- | | | | | | |
| | function | | | | | | |
| | function | | | | | | |
| | allele of | | | | | | |
| | CYP 2C19 or | | | | | | |
| | two reduced | | | | | | |
| | function | | | | | | |
| | alleles. | | | | | | |
| Poor | Individuals | 2.39 | 13.02 | 19.19 | 4.76 | 8.16 | 1.16 |
| Metabolizer: | carrying two | | | | | | |
| PM | loss-of- | | | | | | |
| | function | | | | | | |
| | function | | | | | | |
| | alleles of | | | | | | |
| | CYP2C19. | | | | | | |

* Frequencies and phenotype definitions from https://www.pharmgkb.org/page/cyp2c19RefMaterials, March 2020, except for Japanese column derived from J Clin Pharmacol. 2010 August; 50(8): 929-40

TABLE 8-3
UGT2B17 phenotype definition and expected frequency

| | | Expected Phenotype Frequency (%) * | | | | | |
|---|---|---|---|---|---|---|---|
| | | | East | | South | | Admixed |
| Phenotype | Genotype^{#} | European | Asian | Japan | Asian | African | American |
| Extensive | *1/*1 | 40.95 | 3.37 | 1.92 | 18.00 | 56.88 | 42.36 |
| Metabolizer: | | | | | | | |
| EM | | | | | | | |
| Intermediate | *1/*2 | 44.53 | 27.98 | 21.15 | 43.76 | 37.37 | 46.40 |
| Metabolizer: | | | | | | | |
| IM | | | | | | | |
| Poor | *2/*2 | 14.51 | 68.65 | 76.92 | 38.24 | 5.75 | 11.24 |
| Metabolizer: | | | | | | | |
| PM | | | | | | | |

^{#}*1 in this table denotes absence of UGT2B17*2 allele
* Frequencies from 1000 genomes Phase3v5

TABLE 8-4
Combined UGT2B17 + CYP2C19 expected phenotype
frequency in European ancestry subjects

| | | Expected Combined Phenotype Frequency (%) * | | |
|---|---|---|---|---|
| | | UGT2B17 Phenotype | | |
| | | Extensive | Intermediate | Poor |
| Phenotype | | Metabolizer: EM | Metabolizer: IM | Metabolizer: PM |
| CYP2C19 | Ultra-rapid | 1.92 | 2.08 | 0.68 |
| Phenotype | metabolizer: | | | |
| | UM | | | |
| | Rapid | 11.15 | 12.12 | 3.95 |
| | Metabolizer: | | | |
| | RM | | | |
| | Extensive | 16.20 | 17.61 | 5.74 |
| | Metabolizer: | | | |
| | EM | | | |
| | Intermediate | 10.71 | 11.64 | 3.79 |
| | Metabolizer: | | | |
| | IM | | | |
| | Poor | 0.98 | 1.07 | 0.35 |
| | metabolizer: | | | |
| | PM | | | |

* Derived from CYP2C19 frequencies and phenotype definitions from PharmGKB (https://www.pharmgkb.org/page/cyp2c19RefMaterials) March 2020, UGT2B17 frequencies from 1000 Genomes Phase 3v5

TABLE 8-5
Combined UGT2B17 + CYP2C19 expected phenotype
frequency in East Asian ancestry subjects

| | | Expected Combined Phenotype Frequency (%) * | | |
|---|---|---|---|---|
| | | UGT2B17 Phenotype | | |
| | | Extensive | Intermediate | Poor |
| | | Metabolizer: | Metabolizer: | Metabolizer: |
| Phenotype | | EM | IM | PM |
| CYP2C19 | Ultra-rapid | 0.00 | 0.01 | 0.03 |
| Phenotype | metabolizer: | | | |
| | UM | | | |
| | Rapid | 0.09 | 0.71 | 1.74 |
| | Metabolizer: | | | |
| | RM | | | |
| | Extensive | 1.29 | 10.74 | 26.36 |
| | Metabolizer: | | | |
| | EM | | | |
| | Intermediate | 1.55 | 12.87 | 31.58 |
| | Metabolizer: | | | |
| | IM | | | |
| | Poor | 0.44 | 3.64 | 8.94 |
| | metabolizer: | | | |
| | PM | | | |

* Derived from CYP2C19 frequencies and phenotype definitions from PharmGKB (https://www.pharmgkb.org/page/cyp2c19RefMaterials) March 2020, UGT2B17 frequencies from 1000 Genomes Phase 3v5

TABLE 8-6
Combined UGT2B17 + CYP2C19 expected phenotype
frequency in Japanese ancestry subjects

| | | Expected Combined Phenotype Frequency (%) * | | |
|---|---|---|---|---|
| | | UGT2B17 Phenotype | | |
| | | Extensive | Intermediate | Poor |
| | | Metabolizer: | Metabolizer: | Metabolizer: |
| Phenotype | | EM | IM | PM |
| CYP2C19 | Ultra-rapid | NA | NA | NA |
| Phenotype | metabolizer: | | | |
| | UM | | | |
| | Rapid | NA | NA | NA |
| | Metabolizer: | | | |
| | RM | | | |
| | Extensive | 0.65 | 6.80 | 24.95 |
| | Metabolizer: | | | |
| | EM | | | |
| | Intermediate | 0.97 | 10.16 | 37.27 |
| | Metabolizer: | | | |
| | IM | | | |
| | Poor | 0.38 | 4.03 | 14.78 |
| | metabolizer: | | | |
| | PM | | | |

* Derived from CYP2C19 frequencies from J Clin Pharmacol. 2010 August; 50(8): 929-40, UGT2B17 frequencies from 1000 Genomes Phase 3v5

TABLE 8-7
Combined UGT2B17 + CYP2C19 expected phenotype
frequency in South Asian ancestry subjects

| | | Expected Combined Phenotype Frequency (%) * | | |
|---|---|---|---|---|
| | | UGT2B17 Phenotype | | |
| | | Extensive | Intermediate | Poor |
| | | Metabolizer: | Metabolizer: | Metabolizer: |
| Phenotype | | EM | IM | PM |
| CYP2C19 | Ultra-rapid | 0.53 | 1.28 | 1.12 |
| Phenotype | metabolizer: | | | |
| | UM | | | |
| | Rapid | 3.34 | 8.13 | 7.10 |
| | Metabolizer: | | | |
| | RM | | | |
| | Extensive | 5.32 | 12.93 | 11.30 |
| | Metabolizer: | | | |
| | EM | | | |
| | Intermediate | 7.35 | 17.86 | 15.61 |
| | Metabolizer: IM | | | |
| | Poor | 1.47 | 3.57 | 3.12 |
| | metabolizer: PM | | | |

* Derived from CYP2C19 frequencies and phenotype definitions from PharmGKB (https://www.pharmgkb.org/page/cyp2c19RefMaterials) March 2020, UGT2B17 frequencies from 1000 Genomes Phase 3v5

TABLE 8-8
Combined UGT2B17 + CYP2C19 expected phenotype
frequency in African ancestry subjects

| | | Expected Combined Phenotype Frequency (%) * | | |
|---|---|---|---|---|
| | | UGT2B17 Phenotype | | |
| | | Extensive | Intermediate | Poor |
| | | Metabolizer: | Metabolizer: | Metabolizer: |
| Phenotype | | EM | IM | PM |
| CYP2C19 | Ultra-rapid | 2.44 | 1.60 | 0.24 |
| Phenotype | metabolizer: | | | |
| | UM | | | |
| | Rapid | 13.50 | 8.87 | 1.32 |
| | Metabolizer: | | | |
| | RM | | | |
| | Extensive | 18.79 | 12.34 | 1.84 |
| | Metabolizer: | | | |
| | EM | | | |
| | Intermediate | 19.44 | 12.77 | 1.90 |
| | Metabolizer: IM | | | |
| | Poor | 2.71 | 1.78 | 0.27 |
| | metabolizer: PM | | | |

* Derived from CYP2C19 frequencies and phenotype definitions from PharmGKB (https://www.pharmgkb.org/page/cyp2c19RefMaterials) March 2020, UGT2B17 frequencies from 1000 Genomes Phase 3v5

Expected frequencies of each pairwise phenotype in the United States population were calculated based on the proportion of each race/ethnicity, assuming 60.2% White, 18.3% Hispanic/Latino, 12.3% Black, 4.3% East Asian, 1.3% South Asian, and 3.6% Other. Frequencies were extracted from the American Community Survey Demographic and Housing Estimates 2018 1-Year (U.S. Census Bureau. American Community Survey Demographic and Housing Estimates. (2018)) estimate data profiles. Phenotype frequencies for the “Other” category were assumed to be the average of the phenotype frequencies across all other race/ethic groups.

TABLE 8-9
Combined UGT2B17 + CYP2C19 expected phenotype
frequency in the United States population

| | | Expected Combined Phenotype Frequency (%) * | | |
|---|---|---|---|---|
| | | UGT2B17 Phenotype | | |
| | | Extensive | Intermediate | Poor |
| | | Metabolizer: | Metabolizer: | Metabolizer: |
| Phenotype | | EM | IM | PM |
| CYP2C19 | Ultra-rapid | 1.65 | 1.73 | 0.64 |
| Phenotype | metabolizer: | | | |
| | UM | | | |
| | Rapid | 10.17 | 10.71 | 3.96 |
| | Metabolizer: | | | |
| | RM | | | |
| | Extensive | 16.73 | 17.61 | 6.51 |
| | Metabolizer: | | | |
| | EM | | | |
| | Intermediate | 11.12 | 11.71 | 4.33 |
| | Metabolizer: IM | | | |
| | Poor | 1.28 | 1.34 | 0.50 |
| | metabolizer: PM | | | |

* Derived from CYP2C19 frequencies and phenotype definitions from PharmGKB (https://www.pharmgkb.org/page/cyp2c19RefMaterials) March 2020, UGT2B17 frequencies from 1000 Genomes Phase 3v5, weighted by expected frequencies of each race/ethnic group from the American Community Survey 2018 1-Year estimates

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

1. A method of treating cancer or von Hippel-Lindau (VHL) disease with a safe and effective therapeutic dose of belzutifan in a patient in need thereof, comprising:
(i) determining the belzutifan metabolic status (BMS) of the patient to determine whether the patient has a low metabolizer status, medium metabolizer status, or fast metabolizer status and
(ii) (a) if the patient has the medium or fast metabolizer status, administering belzutifan to the patient at a standard therapeutic dose of 120 mg; or
(ii) (b) if the patient has the low metabolizer status, administering belzutifan, to the patient at a therapeutic dose below that of the standard therapeutic dose.

2. The method of claim 1, wherein the patient has a bodyweight of 45 kg or less.

3. The method of claim 2, wherein:
(a) the patient is determined to have the low metabolizer status when the patient has:
(i) a UGT2B17 PM and CYP2C19 PM phenotype,
(ii) a UGT2B17 PM and CYP2C19 intermediate metabolizer (IM) phenotype; and
(b) the patient is determined to have the medium metabolizer status when the patient has:
(i) a UGT2B17 PM and CYP2C19 extensive metabolizer (EM) phenotype,
(ii) a UGT2B17 PM and CYP2C19 rapid metabolizer (RM) phenotype,
(iii) a UGT2B17 PM and CYP2C19 ultra-rapid metabolizer (UM) phenotype,
(iv) a UGT2B17 IM and CYP2C19 PM phenotype,
(v) a UGT2B17 IM and CYP2C19 IM phenotype,
(vi) a UGT2B17 IM and CYP2C19 EM phenotype,
(vii) a UGT2B17 IM and CYP2C19 RM phenotype,
(viii) a UGT2B17 IM and CYP2C19 UM phenotype,
(ix) a UGT2B17 EM and CYP2C19 PM phenotype,
(x) a UGT2B17 EM and CYP2C19 IM phenotype,
(xi) a UGT2B17 EM and CYP2C19 EM phenotype,
(xii) a UGT2B17 EM and CYP2C19 RM phenotype; or
(xiii) a UGT2B17 EM and CYP2C19 UM phenotype.

4. The method of claim 2, wherein only the phenotype of the UGT2B17 enzyme is determined and
(a) the patient is determined to have the low metabolizer status when the patient has:
a UGT2B17 poor metabolizer (PM) phenotype; and
(b) the patient is determined to have the medium metabolizer status when the patient has:
(i) a UGT2B17 intermediate metabolizer (IM) phenotype, or
(ii) a UGT2B17 extensive metabolizer (EM) phenotype.

5. The method of claim 1, wherein the patient has a bodyweight of 110 kg or more.

6. The method of claim 5, wherein:
(a) the patient is determined to have the low metabolizer status when the patient has a UGT2B17 poor metabolizer (PM) and CYP2C19 PM phenotype;
(b) the patient is determined to have the medium metabolizer status when the patient has:
(i) a UGT2B17 PM and CYP2C19 intermediate metabolizer (IM) phenotype,
(ii) a UGT2B17 PM and CYP2C19 intermediate metabolizer (IM) phenotype,
(iii) a UGT2B17 PM and CYP2C19 extensive metabolizer (EM) phenotype,
(iv) a UGT2B17 PM and CYP2C19 rapid metabolizer (RM) phenotype,
(v) a UGT2B17 PM and CYP2C19 ultra-rapid metabolizer (UM) phenotype,
(vi) a UGT2B17 IM and CYP2C19 PM phenotype,
(vii) a UGT2B17 IM and CYP2C19 IM phenotype,
(viii) a UGT2B17 IM and CYP2C19 EM phenotype,
(ix) a UGT2B17 IM and CYP2C19 RM phenotype,
(x) a UGT2B17 IM and CYP2C19 UM phenotype,
(xi) a UGT2B17 EM and CYP2C19 PM phenotype,
(xii) a UGT2B17 EM and CYP2C19 IM phenotype, or
(xiii) a UGT2B17 EM and CYP2C19 EM phenotype, and
(b) the patient is determined to have the fast metabolizer status when the patient has a
(i) a UGT2B17 EM and CYP2C19 RM phenotype, or
(ii) a UGT2B17 EM and CYP2C19 UM phenotype.

7. The method of claim 1, wherein the patient is being administered a strong inhibitor of UGT2B17 and has a bodyweight of 45 kg or less.

8. The method of claim 7, wherein
(a) the patient is determined to have the low metabolizer status when the patient has:
(i) a CYP2C19 poor metabolizer (PM) phenotype, or
(ii) a CYP2C19 intermediate metabolizer (IM) phenotype; and
(b) the patient is determined to have the medium metabolizer status when the patient has:
(i) a CYP2C19 extensive metabolizer (EM) phenotype,
(ii) a CYP2C19 rapid metabolizer (RM) phenotype, or
(iii) a CYP2C19 ultra-rapid metabolizer (UM) phenotype.

9. The method of claim 1, wherein the patient is being administered a strong UGT2B17 inhibitor and has a bodyweight of greater than 45 kg.

10. The method of claim 9, wherein
(a) the patient is determined to have the low metabolizer status when the patient has:
a CYP2C19 poor metabolizer (PM) phenotype, and
(b) the patient is determined to have the medium metabolizer status when the patient has:
(i) a CYP2C19 intermediate metabolizer (IM) phenotype,
(ii) a CYP2C19 extensive metabolizer (EM) phenotype,
(iii) a CYP2C19 rapid metabolizer (RM) phenotype, or
(iv) a CYP2C19 ultra-rapid metabolizer (UM) phenotype.

11. The method of claim 1, wherein the patient is being administered a strong inhibitor of CYP2C19.

12. The method of claim 11, wherein the patient is under treatment with a CYP2C19 inhibitor selected from fluconazole, fluoxetine, fluvoxamine, or ticlopidine

13. The method of claim 11 er 12, wherein
(a) the patient is determined to have the low metabolizer status when the patient has:
a UGT2B17 poor metabolizer (PM) phenotype; and
(b) the patient is determined to have the medium metabolizer status when the patient has:
(i) a UGT2B17 intermediate metabolizer (IM) phenotype,
(ii) a UGT2B17 extensive metabolizer (EM) phenotype.

14. The method of claim 3, wherein step (i) comprises:
(a) obtaining a biological sample from the patient;
(b) detecting which copies of alleles of UGT2B17 and CYP2C19 are present in the biological sample; and
(c) determining the patient's UGT2B17 and CYP2C19 phenotypes from the detection of the UGT2B17 and CYP2C19 alleles.

15-17. (canceled)

18. The method of claim 14, wherein the patient having the CYP2C19 PM tests positive for two CYP2C19 alleles selected from the group consisting of *2, *3, *4, *5, *6, *7, *8, *9 and *35.

19-21. (canceled)

22. The method of claim 1, wherein in (ii) (b) the therapeutic dose below that of the standard therapeutic dose is 40 mg or 80 mg.

23. The method of claim 1 wherein the patient is in need of treatment of cancer.

24. The method of claim 23, wherein the cancer is renal cell carcinoma.

25. The method of claim 1 wherein the patient is in need of treatment of VHL disease.

26. The method of claim 25, wherein the patient is in need of treatment for VHL disease-associated renal cell carcinoma, central nervous system hemangioblastomas, or pancreatic neuroendocrine tumors, not requiring immediate surgery.
